(19) United States
(12) Patent Application Publication
(10) Pub. No.: US 2010/0036206 A1
(43) Pub. Date: Feb. 11, 2010

(54) Apparatus and method for self-administered maintenance of an ostomy

(76) Inventor: Salvatore J. Lorio, Hicksville, NY (US)

Correspondence Address: Lawrence I. Wechsler;Wechsler & Wechsler, P.C., Suite 9, One Barstow Road, Great Neck, NY, 11021, US

(21) Appl. No.: 12/386,968

(22) Filed: Apr. 24, 2009

Related U.S. Application Data
(60) Provisional application No. 61/125,473, filed on Apr. 25, 2008.

(51) Int. Cl. A61B 1/00

(52) U.S. Cl. 600/247

(57) ABSTRACT

An apparatus is configured to straddle a patient's legs or torso when in a reclined position, and includes a mirror located in a position elevated above a treatment site of a stoma for angularly directing an image as reflected from an overhead vantage of the site to the patient for viewing while in a reclined position.

### CROSS REFERENCE TO RELATED APPLICATIONS

This application claims the benefit of U.S. Provisional Application No. 61/125,473 filed Apr. 25, 2008 entitled APPARATUS AND METHOD FOR SELF-ADMINISTERED MAINTENANCE OF AN OSTOMY.

### BACKGROUND OF THE INVENTION

The present invention relates to an aid for self-administered medical maintenance, and, more particularly, to an apparatus which facilitates the carrying out of unassisted medical maintenance by a user in need of such care on a routine basis and method for its use.

An ostomy is a surgically formed, artificial opening on the abdominal wall through which waste material passes out of the body from the bowel.

A colostomy indicates a type of ostomy in which the opening is from the colon. When the colostomy is in the left colon, only a pad may be needed to cover the opening. However, when the opening is in the right side of the colon, some type of appliance and/or bag is required. There are various types of colostomies.

An ileostomy involves bringing the ileum (the last portion of the small intestine) to the abdominal surface. This new opening on the abdomen is called a stoma. When waste matter reaches the ileum it is liquid, so an appliance is needed for collection of same. Such appliance includes a wafer which is typically changed every three to four days. A pouch, which is also part of the appliance, is generally changed once a day.

Because of the physical location of the stoma, changing the wafer is the most difficult procedure, especially when there is an active stoma (discharging waste). The wafer is required to be placed, centered over the stoma, allowing no more than one-eight of an inch clearance. The stoma can be located on either the left or right side of the abdomen.

At present, people with an ostomy change their wafer and pouch system with at least moderate difficulty. Because of the location of the stoma, looking down trying to center the wafer over the stoma in the standing, sitting or lying down position is not only difficult, inaccurate and awkward, but can also cause neck discomfort or pain. Moreover, such self-practice procedure can be very frustrating with an active stoma. A spouse or other caretaker offering a second pair of hands and eyes is helpful, though embarrassing, and the patient can become dependent upon such assistance. This can be devastating in the event such care becomes unavailable.

Accordingly, it is an object of the invention to provide an apparatus which facilitates unaided performance of the above maintenance procedure by a patient with an ostomy.

It is further object of the invention to provide a method which allows a patient with an ostomy to be independent with the freedom and confidence to apply their wafer and pouch in a comfortable lying down position using two hands, and which allows facilitated control of an active stoma.

It is a further object of the invention to provide an apparatus for assisting the patient in attending to maintenance of the ostomy in a manner which is simple to implement, reliable and cost effective.

### SUMMARY OF THE INVENTION

In accordance with these and other objects of the invention, there is provided an apparatus which straddles a patient's torso when in a reclined position, and which includes a mirror or other means located in a position elevated above a treatment site (site of the stoma) for angularly directing an image as seen from overhead vantage of the site to the patient for viewing.

Briefly stated, an apparatus according to an embodiment of the invention includes a viewing area supportably maintained in an elevated position above a maintenance site of the torso of the patient by bilateral support structure which contacts a support surface on either side of the patient, such as for example, a mattress top. The viewing area is conveniently a suitably sized mirror, a reflection plane of which is oriented at an appropriate angle to reflect the overhead image so that it can be comfortably viewed in the mirror by the patient while in a reclined position.

In order to allow various placements of the apparatus according to the invention above different locations between the head and feet of a patient's body, while still allowing viewability of a desired region to be maintained, in accordance with a preferred embodiment, angular adjustability of the mirror is advantageously provided relative to the patients line of site, conveniently provided by hingable mounting of the mirror to the bilateral support structure. Alternative to hingable mounting, structure allowing incremental angular adjustment by reception of the mirror and/or supporting structure in slots carried on the bilateral supports can be provided for providing similar functional advantage.

In accordance with another advantageous embodiment of the invention, a light source is optionally provided on the apparatus to illuminate the site to be viewed during the maintenance procedure. Such light source is advantageously located in an overhead position relative to the maintenance site, conveniently adjacent to the top of the mirror. The precise type of light source used is not deemed essential to the embodiment, and any illumination device can be employed, including incandescent, fluorescent and/or light emitting diode (LED). However, in this regard, a light with self contained power source (eg. battery) is deemed advantageous, for purposes of facilitated handling and portability.

In accordance with a particularly advantageous embodiment, the apparatus according to the invention is configured in a form which allows the apparatus to be collapsed to a generally flattened state for facilitated storage or portability. This is advantageously achieved by hingable mounting of the bilateral supports to the viewing portion, wherein a hinge axis of the mirror is generally perpendicular to the respective hinge axes of the bilateral supports.

Advantageously, the apparatus is constructed to be as light weight as possible, since it will be handled by the patient alone, at least at times during use, while assuming a reclined posture. This object is achieved by using lightweight material (eg., foamed or honeycombed) or by using stronger material to allow the amounts of materials used for support to be minimized, such as for example, frame-like or cutout configurations.

In practicing a method of maintaining a stoma in accordance with the invention, before positioning an apparatus according to the general guidelines described above, it is advisable for the patient to have all ostomy supplies prepared in advance and in arms reach. The patient gathers such supplies, which advantageously include, for example, a washcloth, soap and water, towel, gauze, tissue paper, barrier past and anything else used during the procedure. The replacement wafer and pouch are removed from the packaging, and the wafer opening is cut to size, if needed. It is noted that wafers can be ordered cut to size once the stoma settles after surgery. Once assuming a reclining position (for example, lying on his/her back), the patient places the bilateral supports of either side such that the apparatus is advantageously straddled just over the knees or in a vicinity thereof. In the case of a folding embodiment of the apparatus, the patient first opens the bilateral support structure of the apparatus, so that the supports are upright relative to structure traversing the patient from above. Once the apparatus is placed in a supported orientation, the hinged lid section with mirror is then lifted and tilted at an angle that gives the best view of the stoma location to the patient. When present, the optional light is then turned on.

Since the apparatus is self supporting on the resting surface, such a mattress top, the patient is free to use both hands to remove the soiled wafer and pouch system. In the lying down position, a patient is afforded more control over an active stoma. After excess paste residue which may be present around the stoma is removed, washed and dried, a new wafer is ready for placement. The release paper on the back of the new wafer is removed, the wafer is positioned in a centered orientation over the stoma, and fixed in position. Once the wafer is fixed, the pouch is attached.

The above, and other objects, features and advantages of the present invention will become apparent from the following description read in conjunction with the accompanying drawings.

### BRIEF DESCRIPTION OF THE DRAWINGS

FIG. 1 is a perspective view of an embodiment of an apparatus according to the invention for facilitated self-maintenance of an ostomy by a patient;

FIG. 2 is a perspective view showing appropriate placement of the apparatus according to FIG. 1 in practicing the method according to the invention;

FIG. 3a-3c are perspective views showing stages in the process for using the apparatus of FIG. 1; and

FIG. 4 is a prototype specification drawing and descriptive functional schematic of an example for manufacture according to an embodiment of the invention.

### DETAILED DESCRIPTION OF THE INVENTION

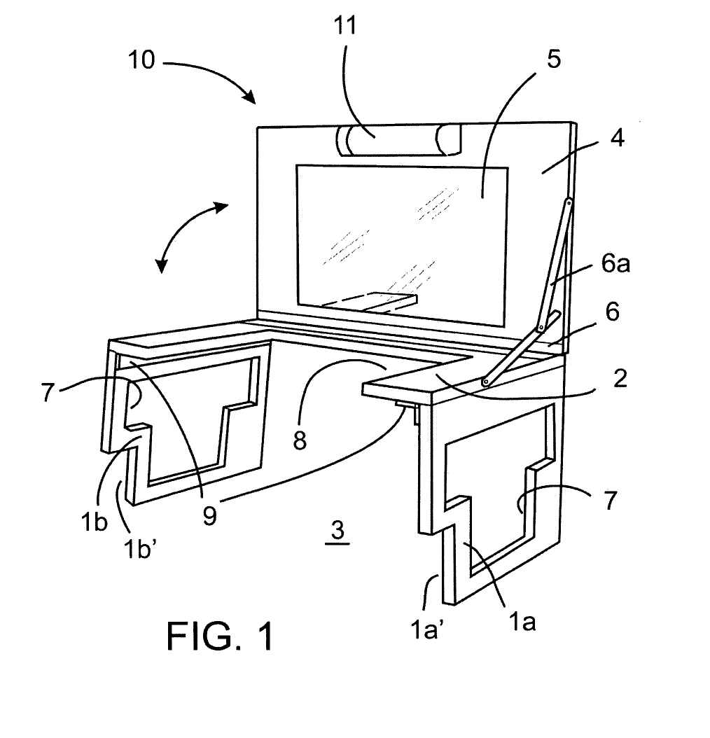

Referring now to the figures, a viewing apparatus according to an embodiment of the invention is depicted generally at 10. Viewing apparatus 10 includes a pair of bilateral supports 1a, 1b, a frame support 2 oriented crosswise to the bilateral supports 1a, 1b, and which is generally horizontal when bilateral supports 1a, 1b are of like height and rest on a level support surface, such as a mattress top 3. A lid portion 4 is also provided, and which is mounted to frame support 2 such that a reflection plane of a mirror 5 will assume an orientation at an acute angle with respect to a plane of the mattress top 3. Advantageously, the acute angular orientation of the reflection plane of the mirror 5 is made to be adjustable by suitable means, for example, in the depicted example by hingable mounting of the lid 4 to the support frame 2 by a hinge 6 (for example, a piano hinge, as shown in FIG. 1). Of course, the selected angular setting selected by the patient will be maintainable in suitable fashion in a fixed position, such as by providing the hinge with sufficient friction to hold the position which is selected as appropriate, or by providing a locking hinge mechanism 6a, as shown. It is further noted that it is alternatively contemplated that the mirror 5 could be mounted directly to the support frame 2, and the lid 4 omitted entirely, or that the mirror 5 could be mounted directly to the bilateral supports 1a, 1b, and the support frame 2 omitted entirely, both without departure from the invention.

Since the viewing apparatus 10 is intended to facilitate unassisted maintenance by the patient alone, weight of the apparatus 10 is advantageously kept as light as possible, for ease of handling, particularly when the patient/user is lying or in another similar reclined position. To this end, suitable approaches for accomplishing minimization of weight can include, for example, cutouts 7, or in the alternative, fabrication of the various structural elements from a material naturally light in weight, for example hollow molded, honeycombed of foamed products.

A further cutout portion 8 is provided in support frame 2 to allow unobstructed viewing of the body region being attended to via the reflecting surface of mirror 5. It will be understood that alternatively, a clear panel could instead be provided if so desired, so long a line of sight for unobstructed viewing of the work area is maintained, and sufficient headroom between the patient and the bottom of the panel (not shown) is present to allow the hands of the user to operate effectively below the panel (not shown).

In the depicted example of FIG. 1, the apparatus 10 is fully foldable for ease of transportation and storage. A pair of hinges 9 conveniently provide hingable movement between support frame 2 and vertical bilateral supports 1a and 1b. To collapse the apparatus 10 for storage when not in use, the bilateral supports 1a, 1b are simply folded inward, to rest against the underside of the support frame 2, and the lid 4 with attached mirror 5 is folded down to contact the top of support frame 2. In the embodiment shown, (advantageously, although not necessarily) the height of each of the bilateral supports 1a, 1b is less than half of the width of the support frame 2, such that they do not overlap one another in a center of the apparatus 10 when in a folded state.

To provide superior illumination of the body site being attended to, a light 11 optionally provided at a desired location on the apparatus 10, conveniently in a top location of lid 4. Cutouts 1a′, 1b′ are provided in bilateral supports 1a, 1b to allow clearance for the light 11 when the apparatus 10 is folded, and the light extends through cutout 8 in the support frame 2.

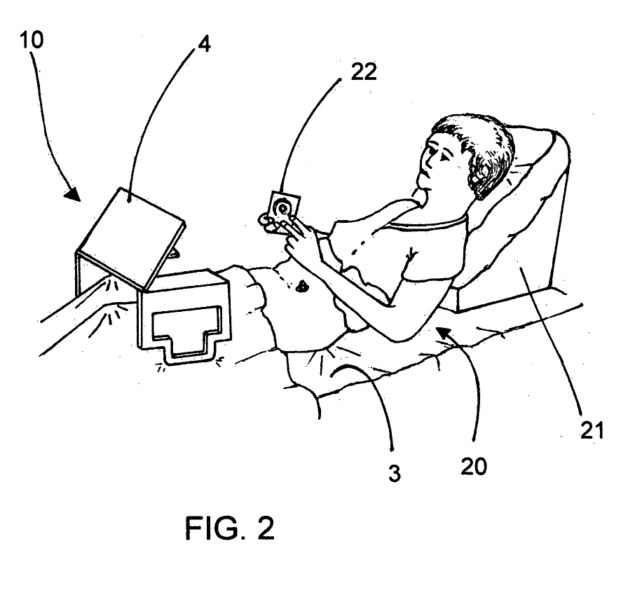

FIG. 2 illustrates a typical positioning of the apparatus 10 according to the invention when prepared for active use. A patient 20 is shown reclined in a lying position with an upper body elevated at an angle by a bolster support 21, which advantageously facilitates viewing of the treatment sight below the apparatus 10. The depicted view of FIG. 2, the patient is shown preparing a fresh replacement wafer 22.

In practicing a method of maintaining a stoma in accordance with the invention, before positioning the apparatus 10 according to the general guidelines described above, it is advisable for the patient to have all ostomy supplies prepared in advance and in arms reach. The patient gathers such supplies, which advantageously include, for example, a washcloth, soap and water, towel, gauze, tissue paper, barrier past and anything else used during the procedure. The replacement wafer and pouch are removed from the packaging, and the wafer opening is cut to size, if needed. It is noted that wafers can be ordered cut to size once the stoma settles after surgery. Once assuming a reclining position (for example, lying on his/her back), the patient places the bilateral supports of either side such that the apparatus is advantageously straddled just over the knees or in a vicinity thereof.

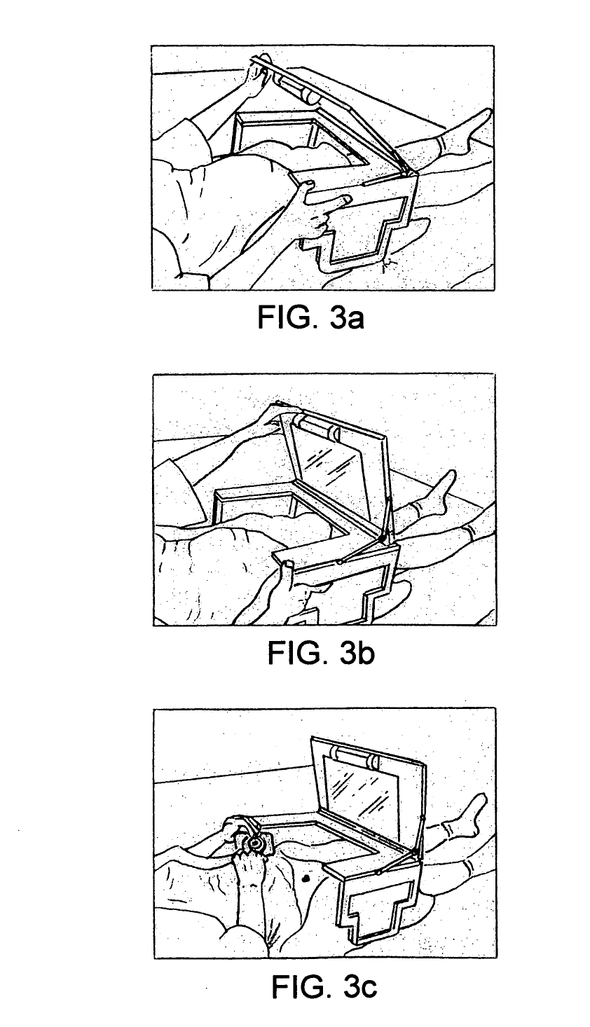

In the case of a folding embodiment of the apparatus, the patient first opens the bilateral support structure of the apparatus, as shown in FIG. 3a, so that the supports are upright relative to structure traversing the patient from above. Once the apparatus is placed in a supported orientation, the hinged lid section with mirror is then lifted and tilted at an angle that gives the best view of the stoma location to the patient, as shown in FIG. 3b. When present, the optional light is then turned on.

Since the apparatus is self supporting on the resting surface, such as a mattress top, the patient is free to use both hands to remove the soiled wafer and pouch system as shown in FIG. 3c. In the lying down position, a patient is afforded more control over an active stoma. After excess paste residue which may be present around the stoma is removed, and the area washed and dried, a new wafer is ready for placement. The release paper on the back of the new wafer is removed, the wafer is positioned in a centered orientation over the stoma, and then fixed in position. Once the wafer is fixed, the pouch is attached.

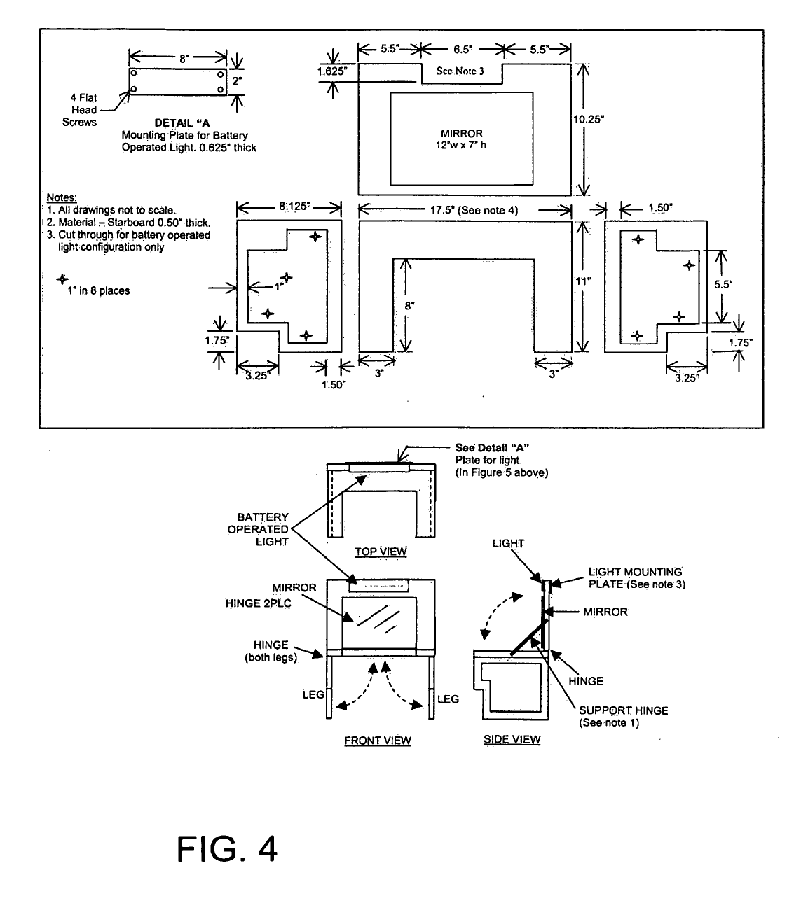

FIG. 4 depicts a design specification (an operative schematic) of a prototype made in accordance with the general guidelines of the invention, showing typical dimensions. However, it will be understood that dimensions will be varied for optional models depending on the physicality of the individual patients using the device disclosed herein, and the invention should not be limited to the precise dimensions depicted in the figure.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention.

What is claimed is:

1. An apparatus for assisting a patient in administering self care to a lower torso region, comprising:
a pair of bilateral supports receivable on a support surface located adjacent to either side of the patient; and
a reflecting surface positionable at an acute angle relative to the support surface, said reflecting surface being mounted the bilateral supports such that said reflecting surface is elevated above the patient and is oriented crosswise to the patient.

2. A method of assisting a patient in administering self care to a lower torso region, comprising:
arranging an apparatus having a pair of bilateral supports on which is mounted a reflecting surface such that said bilateral supports rest on a support surface located adjacent to either side of the patient whereby patient is straddled by the apparatus; and
orienting the reflecting surface at an acute angle relative to the support surface such that a line of sight of the patient provides a view of a site to which the self care is to be administered.
